(19) United States
(12) Patent Application Publication
(10) Pub. No.: US 2023/0136876 A1
(43) Pub. Date: May 4, 2023

(54) ANCHOR INSERTION SYSTEMS FOR A WINGED BONE ANCHOR HAVING A DRIVING CORE

(71) Applicant: Acumed LLC, Hillsboro, OR (US)

(72) Inventors: Steven A. Herbst, Selma, IN (US); John Kent Ellington, Charlotte, NC (US); Jeffrey Alan Greenberg, Indianapolis, IN (US); Charles Mitchell Jobin, Old Greenwich, CT (US); James C. Ritter, Indianapolis, IN (US); Ann Nicole Santich, Portland, OR (US); Mark B. Sommers, Beaverton, OR (US); Brandon Wedam, North Plains, OR (US); Dustin Cluff, St. Helens, OR (US); Steven P. Horst, Dayton, OR (US); Kristopher Scot Lamping, Aloha, OR (US); Kevin The-Kiet Nguyen, Beaverton, OR (US)

(21) Appl. No.: 17/974,965

(22) Filed: Oct. 27, 2022

Related U.S. Application Data
(60) Provisional application No. 63/272,970, filed on Oct. 28, 2021.

(51) Int. Cl. A61B 17/04; A61F 2/08

(52) U.S. Cl. CPC A61B 17/0401; A61F 2/0805; A61F 2/0811; A61B 2017/0409; A61B 2017/0414; A61F 2002/0882; A61F 2002/0888; A61F 2210/0014; A61F 2220/0033; A61F 2230/0093

(57) ABSTRACT

The present disclosure provides new and innovative bone anchors and bone anchor insertion systems that enable smaller bone holes by eliminating the need for a cannula to transport the bone anchor. The provided bone anchors and insertion systems also help prevent damage to suture used in a bone anchor insertion procedure. The bone anchor includes a securement portion that surrounds a core portion. The securement portion includes multiple wings that are shape-set to be splayed outward from the core portion, though may be bent towards or away from the core portion in response to an applied force. The core portion includes a rod extending from a head. The rod includes a drive feature such that a surgeon may engage an inserter with the rod to drive the bone anchor through a bone hole. The bone hole walls maintain the bone anchor in a compressed state.

### PRIORITY CLAIM

This application claims priority to U.S. Provisional Pat. Application No. 63/272,970 filed on Oct. 28, 2021, the entire contents of which are hereby incorporated by reference and relied upon.

### BACKGROUND

Bone anchors are used extensively in open and minimally invasive surgery to reattach tissue to bone. The bone anchor is secured to the bone and one or more sutures attached to the bone anchor are used to secure the tissue to the bone. The tissue can be soft tissue anywhere in the body, for example, a torn rotator cuff in a shoulder or a torn ligament. Typically, in a first step to reattach tissue to bone, a hole is drilled into the bone under arthroscopic visualization. The bone anchor may be inserted through the hole and may be configured to lock itself within the hole in the bone upon deployment therein. Once the bone anchor is secured within the hole in the bone, one or both ends of suture attached to the bone anchor may be tensioned to approximate the positioning of the tissue with respect to the bone. Once the tissue is positioned as desired, the suture may be locked in place to maintain the tension in the suture. The free end or ends of the suture may be clipped under arthroscopic visualization to complete the procedure.

The bone hole size that is drilled when inserting the bone anchor can correlate with a patient’s recovery time. Accordingly, the smallest bone hole that enables achieving fixation needs is desired to reduce recovery times for patients. It is therefore preferred that a bone anchor can be inserted through as small a bone hole as possible while still providing sufficient pull-out strength once installed.

One way to provide such bone anchor properties is a bone anchor having wings constructed with a shape-memory material. The bone anchor may be shape-set in an expanded state with the wings splayed outward, though may be compressed to a smaller size with the wings bent inward as it is translated through a bone hole. Once the bone anchor reaches softer bone (e.g., cancellous bone), the bone anchor returns to its expanded state. In its expanded state, the bone anchor cannot translate back through the bone hole (e.g., cortical bone) because the wings are splayed outward wider than the bone hole.

Typical shape-memory material bone anchors, however, must be inserted through a bone hole with the use of an inserter having a cannula, or may otherwise not have a drive feature to be used with an inserter. For instances with an inserter having a cannula, the bone anchor is positioned within the cannula so that the cannula maintains the bone anchor in a compressed state as it is translated through the bone hole. The bone anchor can then be deployed from the cannula into its expanded state once a desired depth into the bone is reached. The cannula, however, necessitates a larger bone hole than if the bone anchor was inserted into the bone hole on its own, since the cannula surrounds the bone anchor. The larger bone hole may contribute to increased patient recovery times.

Additionally, it is desired to keep suture away from a bone anchor’s shape-memory material wings. For example, the wings may potentially damage the suture by cutting or fraying the suture. Typical bone anchors having shape-memory material wings, however, are not constructed to maintain suture away from its wings.

Accordingly, there is a need for a bone anchor having shape-memory material wings that solves the above drawbacks.

### SUMMARY

The present disclosure provides a new and innovative bone anchor and bone anchor insertion system that enable smaller bone holes in part due to a drive feature of the bone anchor that may be engaged with an inserter.

An example bone anchor includes a core portion including a head and a rod, wherein at least a portion of the core portion is hollow; and a securement portion including a plurality of wings, the securement portion positioned around at least a portion of the rod. The plurality of wings curve away from a central axis of the rod in a rest position and are configured to bend towards and away from the central axis of the rod.

In an aspect of the present disclosure, which may be combined with any other aspect listed herein unless specified otherwise, the securement portion includes four wings.

In an aspect of the present disclosure, which may be combined with any other aspect listed herein unless specified otherwise, the securement portion is constructed of a shape memory material.

In an aspect of the present disclosure, which may be combined with any other aspect listed herein unless specified otherwise, the shape memory material is nitinol.

In an aspect of the present disclosure, which may be combined with any other aspect listed herein unless specified otherwise, the plurality of wings are configured to bend towards the central axis of the rod such that the plurality of wings are substantially parallel to the central axis of the rod.

In an aspect of the present disclosure, which may be combined with any other aspect listed herein unless specified otherwise, when the plurality of wings are substantially parallel to the central axis of the rod, the securement portion surrounds the entire length of the rod.

In an aspect of the present disclosure, which may be combined with any other aspect listed herein unless specified otherwise, the hollow portion of the core portion is a notch in an end of the rod opposite the head.

In an aspect of the present disclosure, which may be combined with any other aspect listed herein unless specified otherwise, the rod includes an opening with a central axis perpendicular to the central axis of the rod.

In an aspect of the present disclosure, which may be combined with any other aspect listed herein unless specified otherwise, the core portion and the securement portion are separate components.

In an aspect of the present disclosure, which may be combined with any other aspect listed herein unless specified otherwise, the core portion and the securement portion are press fit together.

In an aspect of the present disclosure, which may be combined with any other aspect listed herein unless specified otherwise, the core portion and the securement portion are integrally formed as a single component.

In an aspect of the present disclosure, which may be combined with any other aspect listed herein unless specified otherwise, the securement portion further includes a base from which the plurality of wings extend, and wherein the base contacts the head.

In an aspect of the present disclosure, which may be combined with any other aspect listed herein unless specified otherwise, the base contacts the head on a first side of the base and the plurality of wings extend from a second side of the base, the first side being opposite the second side.

In an aspect of the present disclosure, which may be combined with any other aspect listed herein unless specified otherwise, each of the plurality of wings is equally dispersed around the central axis of the rod.

In some examples, an example bone anchor insertion system includes: an inserter including an insertion end having a key; suture material; and a bone anchor including: a core portion having a head and a rod, wherein the rod includes an opening perpendicular to a central axis of the rod and a drive notch parallel with the central axis of the rod; and a securement portion including a plurality of wings, the securement portion positioned around at least a portion of the rod. The plurality of wings curve away from the rod’s central axis in a rest position and are configured to bend towards and away from the rod’s central axis. The suture material is positioned through the opening in the rod. The key of the inserter is configured to be positioned within the drive notch of the rod so that the inserter drives the bone anchor through a bone canal.

In an aspect of the present disclosure, which may be combined with any other aspect listed herein unless specified otherwise, the inserter and the bone anchor are configured such that the bone anchor is driven through a bone canal via the inserter without a cannula.

In an aspect of the present disclosure, which may be combined with any other aspect listed herein unless specified otherwise, the key and the drive notch are correspondingly configured such that the inserter and the bone anchor are prevented from rotating with respect to one another when the key is positioned within the drive notch.

In some examples, an example bone anchor insertion system includes an inserter having an insertion end with a tapered groove; suture material; and a bone anchor including: a core portion including a head and a rod, wherein at least a portion of the core portion is hollow; and a securement portion including a plurality of wings, the securement portion positioned around at least a portion of the rod. The plurality of wings curve away from the rod’s central axis in a rest position and are configured to bend towards and away from the rod’s central axis. The inserter’s insertion end is configured to be positioned within the rod so that the bone anchor is driven through a bone canal via the inserter. The suture material is positioned through the rod and within the tapered groove.

In an aspect of the present disclosure, which may be combined with any other aspect listed herein unless specified otherwise, the head includes two separate components joined by a bar.

In an aspect of the present disclosure, which may be combined with any other aspect listed herein unless specified otherwise, the rod is configured to maintain the suture material within the plurality of wings as the bone anchor is driven through the bone canal via the inserter.

### BRIEF DESCRIPTION OF THE DRAWINGS

FIG. 1 illustrates a perspective view of a bone anchor having a hollow core portion, according to an aspect of the present disclosure.

FIG. 2 illustrates a perspective view of a securement portion, according to an aspect of the present disclosure.

FIG. 3A illustrates a perspective view of a core portion, according to an aspect of the present disclosure.

FIG. 3B illustrates a perspective view of a core portion having a head with a bar, according to an aspect of the present disclosure.

FIG. 4 illustrates a portion of a bone anchor insertion system having a hollow core portion and an inserter with a tapered groove, according to an aspect of the present disclosure.

FIG. 5 illustrates a portion of a bone anchor insertion system having a core portion with a drive notch and an inserter with a key, according to an aspect of the present disclosure.

FIG. 6 illustrates a perspective view of a bone anchor insertion system, according to an aspect of the present disclosure.

FIGS. 7A to 7C illustrate perspective views of a method for inserting a bone anchor with a bone anchor insertion system, according to an aspect of the present disclosure.

FIG. 8 illustrates a perspective view of a securement portion including a base with a reduced section, according to an aspect of the present disclosure.

FIG. 9 illustrates a side view of the securement portion of FIG. 8, according to an aspect of the present disclosure.

FIG. 10 illustrates a cross-sectional view of the securement portion of FIG. 9, according to an aspect of the present disclosure.

### DETAILED DESCRIPTION

The present disclosure provides new and innovative bone anchors and bone anchor insertion systems that enable smaller bone holes by providing a bone anchor having a drive feature that may be engaged with an inserter. The provided bone anchors and insertion systems also help prevent damage to suture used in a bone anchor insertion procedure. The presently disclosed bone anchor includes a core portion and a securement portion that surrounds the core portion. The securement portion includes multiple wings. The wings are curved or splayed outward from the core portion at rest, though may be bent towards or away from the core portion in response to an applied force. For instance, as the bone anchor is driven into a bone hole that is narrower than the outward perimeter of the splayed wings, the bone hole walls force the splayed wings to bend towards the core portion to a compressed state to fit within the bone hole. Once the force is insufficient to maintain the wings in the compressed state (e.g., softer bone), the wings return to their initial, splayed outward state.

The core portion includes a rod extending from a head. The securement portion is positioned around the core portion’s rod. The rod extends between the multiple wings such that the wings are dispersed about the rod’s central axis. The core portion’s rod is configured such that it may be used as a drive feature with an insertion instrument (e.g., inserter). Stated differently, a surgeon may engage an inserter with the rod to drive the bone anchor through a bone hole. A cannula is not needed to transport the bone anchor. Moreover, a cannula is not needed to maintain the bone anchor in a compressed state. Rather, while the surgeon drives the bone anchor through the bone hole, the bone anchor is maintained in a compressed state by the bone hole itself. Accordingly, smaller bone holes may be drilled by eliminating the need for an inserter with a cannula to deploy a bone anchor, which may help contribute to decreased patient recovery times.

In some examples, the core portion may include a notch on its end opposite the head. The notch shape matches the shape of an inserter tip such that the tip may be positioned within the notch. This enables the bone anchor to be driven head first via the inserter. The notch may have at least one flat side to prevent rotation with respect to the inserter. In such notch examples, the core portion may include an opening perpendicular to its central axis. Suture may be positioned through the opening. The suture may be secured to the inserter while transporting the bone anchor, which couples the bone anchor to the inserter. The opening may be positioned on the rod near the notch end (e.g., as close as possible) to prevent the suture from contacting the wings as much as possible. This configuration may help protect the suture from potential damage by the wings.

In other examples, instead of a notch, at least the core portion’s rod may be hollow along its entire length. The core portion’s head may be hollow and/or may include a suture securing structure. A tip of an inserter may be positioned within the end of the rod opposite the head. This enables the bone anchor to be driven head first via the inserter. In such examples, suture may be secured at the core portion’s head (e.g., a knot or other structure in the core’s head) and positioned through the rod. The suture may be secured to the inserter while transporting the bone anchor, which couples the bone anchor to the inserter. Positioning the suture through the rod helps maintain the suture away from the wings and therefore helps protect the suture from potential damage by the wings. Additionally, in such examples, the inserter’s tip may have a tapered groove that enables the tip to be inserted within the rod while also enabling the suture to exit the rod.

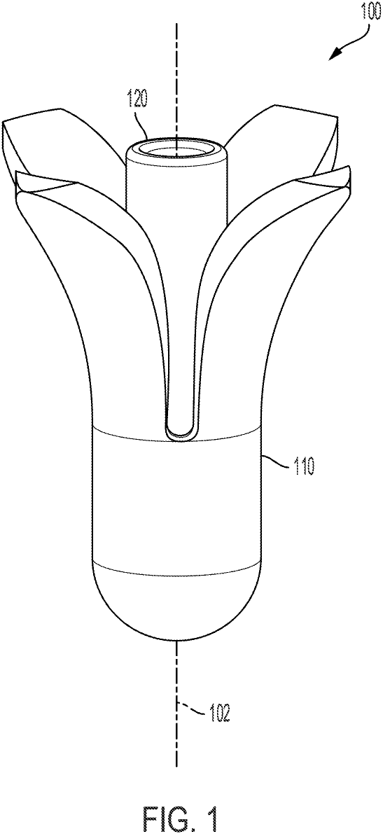

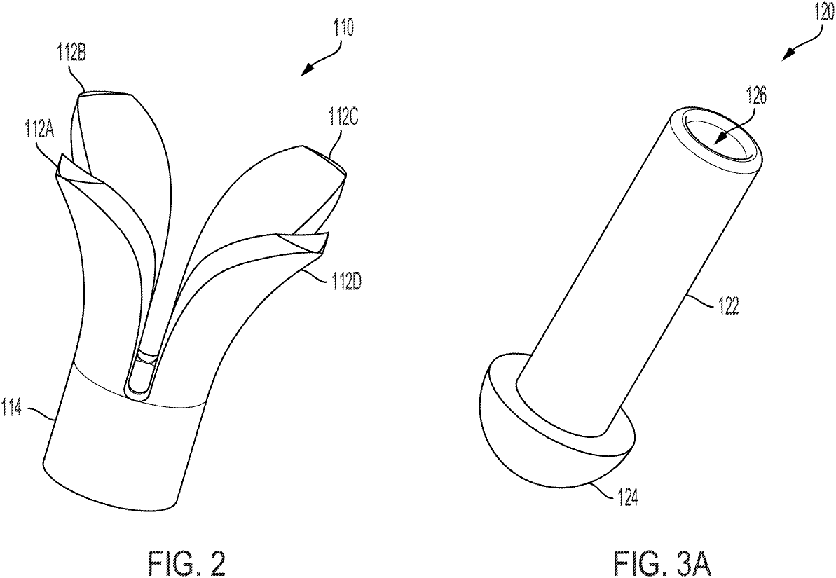

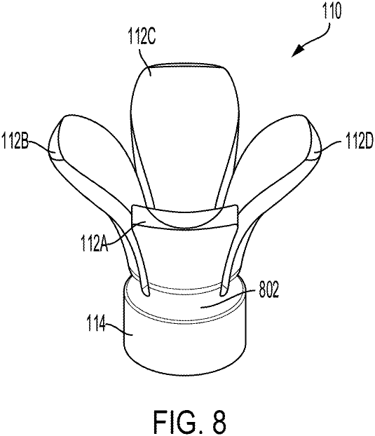

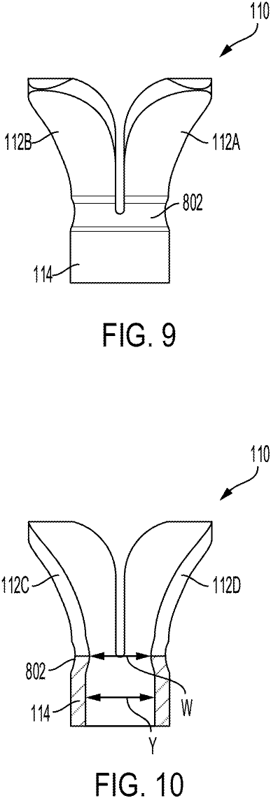

FIGS. 1, 2, and 3A illustrate perspective views of a securement portion 110 and a core portion 120 of an example bone anchor 100. The bone anchor 100 includes the securement portion 110 and the core portion 120. The securement portion 110 is positioned around the core portion 120. In various instances, such as the illustrated example, the securement portion 110 and the core portion 120 are separate components. The securement portion 110 may be press fit (e.g., FIGS. 8-10) or laser welded to the core portion 120. In other instances, the securement portion 110 and the core portion 120 may be machined as a single component.

The securement portion 110 includes wings 112A, 112B, 112C, 112D that extend from a base 114. The base 114 is in contact with the head 124 of the core portion 120. The wings 112A, 112B, 112C, 112D are dispersed about a central axis 102 of the rod 122 of the core portion 120. In some aspects, the wings 112A, 112B, 112C, 112D may be equally dispersed about the central axis 102. In some instances, the securement portion 110 may include four wings 112A, 112B, 112C, 112D as illustrated. In other instances, the securement portion 110 may include more or less than four (e.g., two, three, five, six) wings 112A, 112B, 112C, 112D.

The securement portion 110 may be constructed of any suitable medical-grade material that enables the flexibility of the wings 112A, 112B, 112C, 112D described herein. For instance, the wings 112A, 112B, 112C, 112D or the securement portion 110 as a whole may be constructed of a shape-memory material, such as nitinol. In such instances, the wings 112A, 112B, 112C, 112D may be shape-set to splay or curve away from the central axis 102.

As illustrated, the wings 112A, 112B, 112C, 112D splay or curve away from the central axis 102 while at rest. The wings 112A, 112B, 112C, 112D are flexible and may bend towards and away from the central axis 102 in response to an applied force. For example, the wings 112A, 112B, 112C, 112D may bend towards the central axis 102 such that the wings 112A, 112B, 112C, 112D are substantially parallel with the central axis 102 when the bone anchor 100 is driven into a bone hole. When the wings 112A, 112B, 112C, 112D are substantially parallel to the central axis 102, the wings 112A, 112B, 112C, 112D may extend past the end of the rod 120. In other instances, the wings 112A, 112B, 112C, 112D may be shorter such that they terminate at the end of the rod 120 or terminate along the rod 120 prior to its end. The wings 112A, 112B, 112C, 112D may also bend away from the central axis 102 in response to an applied force, further splaying away from the central axis 102. In either instance, when the applied force is removed, the wings 112A, 112B, 112C, 112D return to the illustrated example rest position. The wings 112A, 112B, 112C, 112D may be splayed a greater or lesser amount from the central axis 102 in the rest position in other examples.

In some aspects of the present disclosure, each of the wings 112A, 112B, 112C, 112D is configured the same as the other wings 112A, 112B, 112C, 112D. In such aspects, each of the wings 112A, 112B, 112C, 112D has the same shape, an equal length, is splayed the same amount from the central axis 102, and requires the same amount of applied force to bend towards or away from the central axis 102.

In other aspects of the present disclosure, one or more of the wings 112A, 112B, 112C, 112D may be different than the other wings 112A, 112B, 112C, 112D. For example, one or more wings 112A, 112B, 112C, 112D may have a different length. In another example, one or more wings 112A, 112B, 112C, 112D may be shaped differently. In another examples, one or more wings 112A, 112B, 112C, 112D may be splayed a greater or lesser amount from the central axis 102. In another example, one or more wings 112A, 112B, 112C, 112D may be stronger than the others such that a greater applied force is needed to bend the one or more stronger wings 112A, 112B, 112C, 112D toward or away from the central axis 102. In another example, one or more wings 112A, 112B, 112C, 112D may be weaker than the others such that less applied force is needed to bend the one or more stronger wings 112A, 112B, 112C, 112D toward or away from the central axis 102. For instance, the width of a particular wing 112A, 112B, 112C, 112D contributes to its strength such that a greater width corresponds to greater strength.

The one or more different wings 112A, 112B, 112C, 112D may cause the bone anchor 100 to apply unequal or asymmetric force on a bone. Since many bone surfaces are not flat, the unequal or asymmetric force may be beneficial in certain instances because it may enable the bone anchor 100 to better conform to an uneven surface as compared to a bone anchor that applies symmetric force.

In some aspects, as mentioned above, the securement portion 110 and the core portion 120 may be press fit together. FIGS. 8 to 10 illustrate an example securement portion 110 having a base 114 formed with a reduced section 802 that is used to press fit the securement portion 110 to the core portion 120. As shown in the cross-section of FIG. 10, a width W of the interior of the base 114 at the reduced section 802 is smaller than a width Y of the interior of the base 114 outside of the reduced section 802. In this way, the base 114 may press against the rod 122 of the core portion 120 at the reduced section 802 when the rod 122 is positioned through the securement portion 110 to thereby press fit the securement portion 110 and the core portion 120 together. In an example, the securement portion 110 may be heat-treated and/or crimped to form the reduced section 802. In some instances, the rod 122 of the core portion 120 may be positioned through the securement portion 110 when the securement portion 110 is heat-treated and/or crimped. In other instances, the securement portion 110 may be heat-treated and/or crimped prior to positioning the rod 122 through the securement portion 110.

The core portion 120 of the example bone anchor 100 includes a rod 122 and a head 124. The rod 122 and the head 124 may be formed integrally with one another or may be separate components that are connected. The core portion 120 may be constructed of any suitable medical-grade material, such as titanium, stainless steel or a medical-grade plastic (e.g., polyether ether ketone).

At least a portion of the core portion 120 is hollow. In some aspects, such as the ones illustrated in FIGS. 1 to 4, the core portion 120 includes a channel 126 that opens on one end of the rod 122. The channel 126 extends the length of the rod 122. In some instances, the channel 126 extends all the way through the head 124 such that it opens at the outermost end of the head 124. In such instances, suture may be positioned through the channel 126 of the core portion 120 and secured (e.g., knotted) to the core portion 120 outside of the head 124.

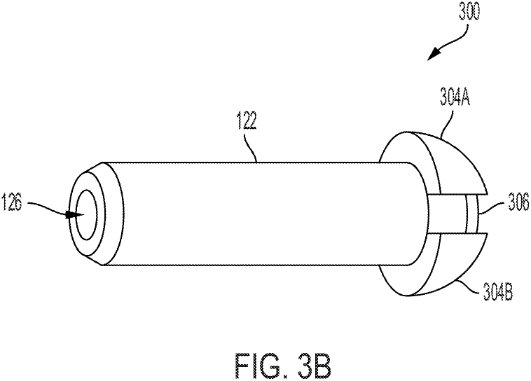

In other instances, the channel 126 connects to a different opening in the head 124. In such other instances, the head 124 may be configured to secure suture to the core portion 120 within the head 124. For example, FIG. 3B illustrates an example core portion 300 that includes a head that is split into a first head part 304A and a second head part 304B. A rod 122 having a channel 126 extends from the first and second head parts 304A and 304B. A bar 306 connects the first head part 304A to the second head part 304B. Suture positioned through the channel 126 may be positioned around the bar 306 and back through the channel 126 to secure the suture to the core portion 300.

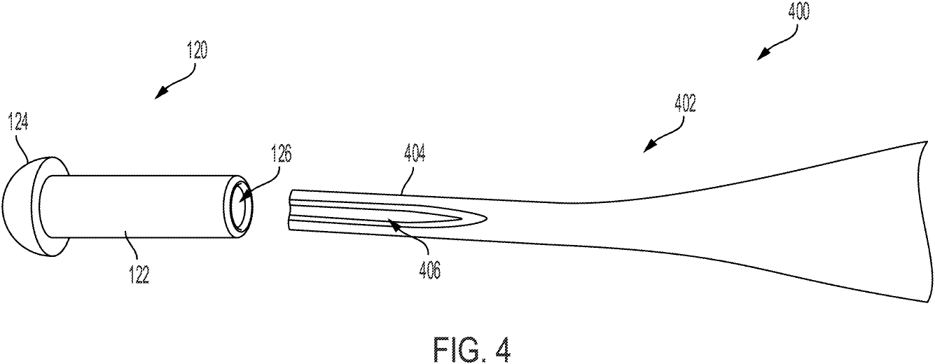

The channel 126 of the core portion 120 may act as a drive feature in connection with an inserter. FIG. 4 illustrates a portion of an example bone anchor insertion system 400 including an inserter 402 and the core portion 120 of the example bone anchor 100. It should be appreciated that the securement portion 110 is not shown around the core portion 120 merely for illustrative purposes. The inserter 402 includes an insertion tip 404. The inserter 402 may also include a shaft (not illustrated) and handle (not illustrated). The insertion tip 404 is configured such that it may be inserted within the channel 126 of the bone anchor 120. The insertion tip 404 may be tapered, with its end being the most narrow portion, so that the insertion tip 404 may only be positioned within the channel 126 a certain amount (e.g., the end does not reach the head 124). The insertion tip 404 may also include a tapered groove 406. The tapered groove 406 enables suture to exit the channel 126 while the insertion tip 404 is inserted within the channel 126. When the insertion tip 404 is positioned within the channel 126, at least a portion of the tapered groove 406 remains outside of the channel 126 to that the suture may exit the channel 126.

When the insertion tip 404 is inserted within the channel 126, the suture may be secured to the inserter 402, which couples the bone anchor 120 to the inserter 402. A surgeon may drive the coupled bone anchor 120 through a bone hole via the inserter 402. The surgeon may deploy the suture from the inserter 402 and remove the insertion tip 404 from the channel 126 to decouple the inserter 402 from the bone anchor 120. Additionally, the rod 122 of the core portion 120 helps protect the suture from accidental damage by the wings (not illustrated) since the suture is positioned within the rod 122.

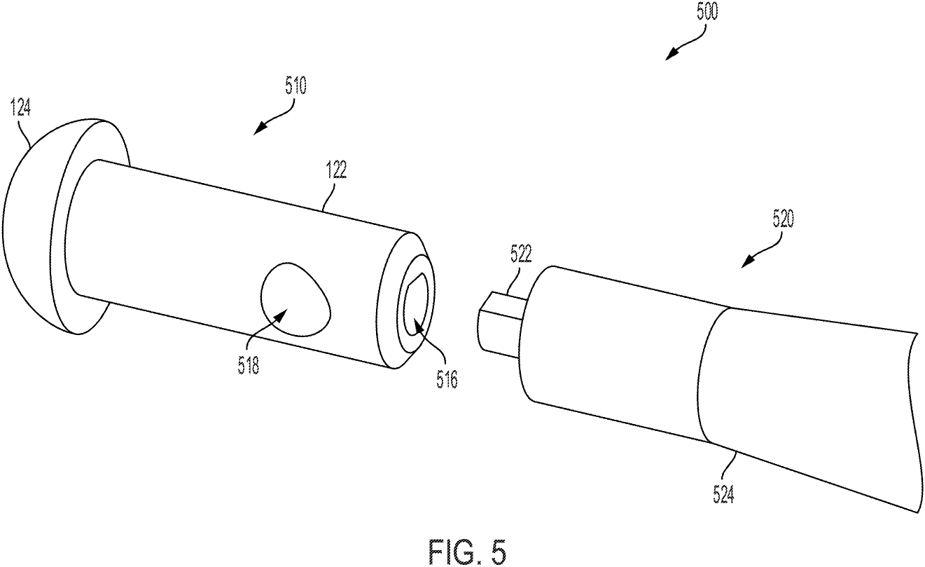

In some aspects of the present disclosure, the hollow part of the core portion may be a notch instead of a channel. For example, FIG. 5 illustrates a portion of a bone anchor insertion system 500 having a core portion 510 with a notch 516. It should be appreciated that a securement portion (e.g., the securement portion 110) is not shown around the core portion 510 merely for illustrative purposes. The bone anchor insertion system 500 may also include suture (not illustrated). In this example, the core portion 510 includes a rod 122 extending from a head 124. The notch 516 is an absence of material that extends into the end of the rod 122 as illustrated. The core portion 510 may include an opening 518 through the rod 122. The notch 516 may end prior to reaching the opening 518 in various instances. Due to the absence of a channel in the example core portion 510, suture may instead be positioned through the opening 518 to secure the suture to the core portion 510.

The bone anchor insertion system 500 also includes an example inserter 520. The inserter 520 includes a key 522 on its insertion end. The inserter 520 also includes a shaft 524 and may include a handle (not illustrated). The notch 516 acts as a drive feature for use with the inserter 520. The key 522 and the notch 516 are configured to correspond to one another such that the key 522 may be inserted within the notch 516. In some instances, the key 522 and the notch 516 may include at least one corresponding flat side in order to prevent rotation of the core portion 510 with respect to the inserter 520 or vice versa.

FIG. 6 illustrates a perspective view of the bone anchor insertion system 500 showing a bone anchor 602 that includes a securement portion 110 surrounding the core portion 510. The suture 606 of the bone anchor system 500 is also illustrated. The inserter 520 is shown engaged with the bone anchor 602 such that the key 522 is positioned within the notch 516. In this position, the suture 606 placed through the opening 518 may be secured to the inserter 520, which couples the bone anchor 602 to the inserter 520. In the example bone anchor insertion system 500, the suture 606 is not positioned within the rod 122. Nevertheless, the opening 518 may be positioned near the end of the rod 122 closest the inserter 520 to help maintain the suture 606 away from the wings 112A, 112B, 112C, 112D. This configuration helps prevent the wings 112A, 112B, 112C, 112D from accidentally damaging the suture 606.

When the key 522 is inserted within the notch 516 and the suture 606 couples the bone anchor 602 to the inserter 520 as illustrated, a surgeon may drive the coupled bone anchor 602 through a bone hole via the inserter 520. The surgeon may thereafter deploy the suture 606 from the inserter 520 and translate the inserter 520 away from the bone anchor 602 to decouple the inserter 520 from the bone anchor 602.

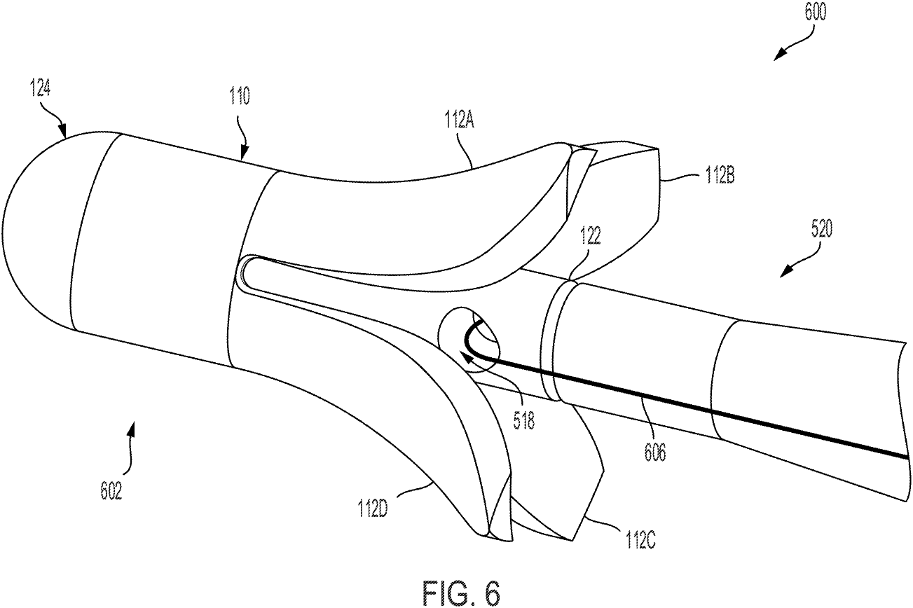

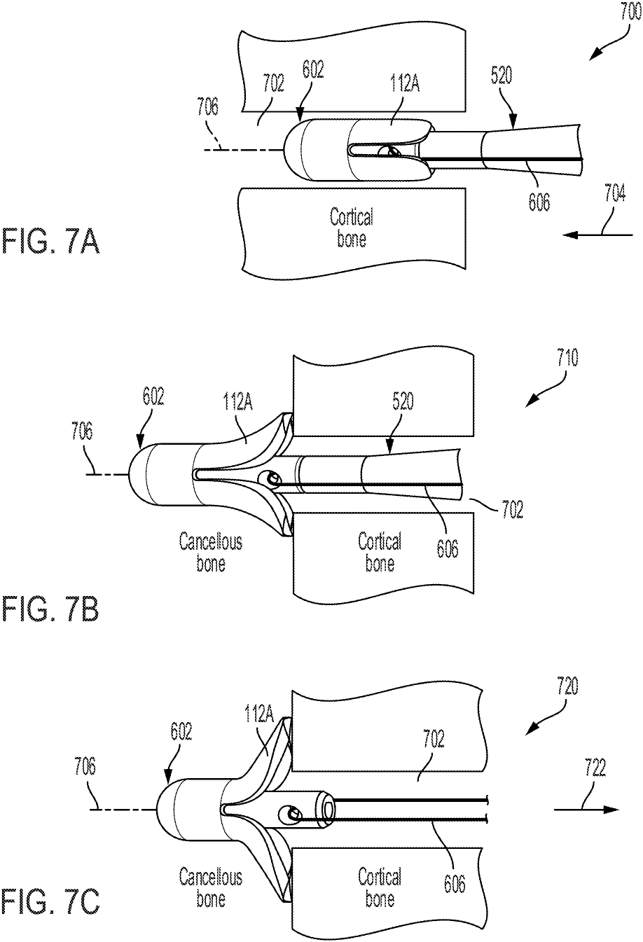

FIGS. 7A to 7C illustrate perspective views of example processes for inserting the bone anchor 602 through a bone hole 702 using the inserter 520. The example process 700 of FIG. 7A illustrates the inserter 520 coupled to the bone anchor 602 as a surgeon drives the bone anchor 602 through a bone hole 702 in the direction of the arrow 704. The bone hole 702 may be created by drilling into cortical bone to a length that is longer than the length of the bone anchor 602. A gap is illustrated between the walls of the bone hole 702 and the bone anchor 602 solely for the sake of clarity. As the surgeon drives the bone anchor 602 into and through the bone hole 702, the outer surface of the bone anchor 602 contacts the walls of the bone hole 702, which causes the wings 112A-112D of the securement portion 110 to fold up or bend towards the central axis 706 as illustrated. The hardness of cortical bone is sufficient to effect an opposing force that maintains the wings 112A-112D of the securement portion 110 in this folded up or compressed state.

Once the surgeon drives the bone anchor 602 into softer bone, such as cancellous bone, however, the hardness of the cancellous bone is insufficient to effect such an opposing force. The wings 112A-112D of the securement portion 110 accordingly splay away from the central axis 706, into the cancellous bone, to return to their resting position, as illustrated in the example process 710 of FIG. 7B. In some instances, once the surgeon detects that the wings 112A-112D of the securement portion 110 have returned to their resting position, the surgeon may deploy the suture 606 from the inserter 520. The surgeon may then decouple the inserter 520 from the bone anchor 602 by translating the inserter 520 out of the bone hole 702.

Once the inerter 520 is removed, the surgeon may tension the suture 606 in the direction of the arrow 722, as illustrated in the example process 720 of FIG. 7C. As the surgeon tensions the suture 606 in the direction of the arrow 722 from the positioning illustrated in FIG. 7B, the suture 606 pulls the bone anchor 602 in the direction of the arrow 722. The wings 112A-112D of the securement portion 110, however, are prevented from translating in the direction of the arrow 722 by the cortical bone. Instead, the cortical bone causes the wings 112A-112D of the securement portion 110 to splay or bend further away from the central axis 706. Once the wings 112A-112D of the securement portion 110 splay or bend apart a sufficient amount to provide an amount of pull-out strength that the surgeon desires, the surgeon may lock the suture 606 in place. The surgeon may then clip the free ends of the suture 606 and complete the procedure.

In other instances, the surgeon may position the bone anchor 602, causing the wings 112A-112D of the securement portion 110 to splay outward, prior to deploying the suture 606 from the inserter 520. In such other instances, the inserter 520 remains coupled to the bone anchor 602 prior to deploying the suture 606 so translating the inserter 520 in the direction of the arrow 722 has the same effect as tensioning the suture 606 in the direction of the arrow 722 as described above. Once the bone anchor 602 is positioned to the surgeon’s liking, the surgeon may deploy the suture 606 while maintaining tension in the suture 606. The surgeon may then lock the suture 606 in place and clip its free ends.

The presently disclosed bone anchor insertion system therefore enables smaller bone holes as compared to typical bone anchor insertion systems by eliminating the need for a cannula to transport a bone anchor. Rather than a cannula, the presently disclosed bone anchor insertion system includes a bone anchor with a drive feature that enables a surgeon to engage an inserter to the bone anchor to drive the bone anchor through a bone hole, which maintains the bone anchor in a folded up or compressed state (e.g., FIG. 7A). The provided bone anchor is also configured to help prevent accidental damage to the suture.

In the present disclosure, the terms “including” and “comprising” are used in an open-ended fashion, and thus should be interpreted to mean “including, but not limited to....” As used herein, “about,” “approximately” and “substantially” are understood to refer to numbers in a range of numerals, for example the range of -10% to +10% of the referenced number, preferably -5% to +5% of the referenced number, more preferably -1% to +1% of the referenced number, most preferably -0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

It is to be understood that at least some of the figures and descriptions herein have been simplified to illustrate elements that are relevant for a clear understanding of the disclosure while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements may not be provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term ‘at least one of X or Y’ or ‘at least one of X and Y’ should be interpreted as X, or Y, or X and Y.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

The invention is claimed as follows:

1. A bone anchor comprising:
a core portion including a head and a rod, wherein at least a portion of the core portion is hollow; and
a securement portion including a plurality of wings, the securement portion positioned around at least a portion of the rod,
wherein the plurality of wings curve away from a central axis of the rod in a rest position and are configured to bend towards and away from the central axis of the rod.

2. The bone anchor of claim 1, wherein the securement portion includes four wings.

3. The bone anchor of claim 1, wherein the securement portion is constructed of a shape memory material.

4. The bone anchor of claim 3, wherein the shape memory material is nitinol.

5. The bone anchor of claim 1, wherein the plurality of wings are configured to bend towards the central axis of the rod such that the plurality of wings are substantially parallel to the central axis of the rod.

6. The bone anchor of claim 5, wherein when the plurality of wings are substantially parallel to the central axis of the rod, the securement portion surrounds the entire length of the rod.

7. The bone anchor of claim 1, wherein the hollow portion of the core portion is a notch in an end of the rod opposite the head.

8. The bone anchor of claim 1, wherein the rod includes an opening with a central axis perpendicular to the central axis of the rod.

9. The bone anchor of claim 1, wherein the core portion and the securement portion are separate components.

10. The bone anchor of claim 9, wherein the core portion and the securement portion are press fit together.

11. The bone anchor of claim 1, wherein the core portion and the securement portion are integrally formed as a single component.

12. The bone anchor of claim 1, wherein the securement portion further includes a base from which the plurality of wings extend, and wherein the base contacts the head.

13. The bone anchor of claim 12, wherein the base contacts the head on a first side of the base and the plurality of wings extend from a second side of the base, the first side being opposite the second side.

14. The bone anchor of claim 1, wherein each of the plurality of wings is equally dispersed around the central axis of the rod.

15. A bone anchor insertion system comprising:
an inserter including an insertion end having a key;
suture material; and
a bone anchor including:
a core portion having a head and a rod, wherein the rod includes an opening perpendicular to a central axis of the rod and a drive notch parallel with the central axis of the rod; and
a securement portion including a plurality of wings, the securement portion positioned around at least a portion of the rod,
wherein the plurality of wings curve away from the rod’s central axis in a rest position and are configured to bend towards and away from the rod’s central axis,
wherein the suture material is positioned through the opening in the rod, and
wherein the key of the inserter is configured to be positioned within the drive notch of the rod so that the inserter drives the bone anchor through a bone canal.

16. The bone anchor insertion system of claim 15, wherein the inserter and the bone anchor are configured such that the bone anchor is driven through a bone canal via the inserter without a cannula.

17. The bone anchor insertion system of claim 15, wherein the key and the drive notch are correspondingly configured such that the inserter and the bone anchor are prevented from rotating with respect to one another when the key is positioned within the drive notch.

18. A bone anchor insertion system comprising:
an inserter having an insertion end with a tapered groove;
suture material; and
a bone anchor including:
a core portion including a head and a rod, wherein at least a portion of the core portion is hollow; and
a securement portion including a plurality of wings, the securement portion positioned around at least a portion of the rod,
wherein the plurality of wings curve away from the rod’s central axis in a rest position and are configured to bend towards and away from the rod’s central axis,
wherein the inserter’s insertion end is configured to be positioned within the rod so that the bone anchor is driven through a bone canal via the inserter, and
wherein the suture material is positioned through the rod and within the tapered groove.

19. The bone anchor insertion system of claim 18, wherein the head includes two separate components joined by a bar.

20. The bone anchor insertion system of claim 18, wherein the rod is configured to maintain the suture material within the plurality of wings as the bone anchor is driven through the bone canal via the inserter.
